SEQUENCE LISTING (3 sequences)

SEQ ID NO: 1 - DNA, 20 bp, Artificial Sequence
  F27
agagtttgatcmtggctcag

SEQ ID NO: 2 - DNA, 19 bp, Artificial Sequence
  1492R
ggytaccttgttacgactt

SEQ ID NO: 3 - DNA, 1386 bp, Beijerinckia fluminesis
gcgggcggcaggcttacacatgcagtcgaacgccccgcaaggggagtggcagacgggtgagtaacgcgtgggaacataccctttcctgcggaatagctccgggaaactggaattaataccgcatacgccctacgggggaaagatttatcggggaaggattggcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccttagggttgtaaagctctttcaccgatgaagataatgacggtagtcggagaagaagccccggctaacttcgtgccagcagccgcggtaatacgaagggggctagcgttgttcggaattactgggcgtaaagcgcacgtaggcggatatttaagtcaggggtgaaatcccgcagctcaactgcggaactgcctttgatactgggtatcttgagtatggaagaggtaagtggaattccgagtgtagaggtgaaattcgtagatattcggaggaacaccagtggcgaaggcggcttactggtccattactgacgctgaggtgcgaaagcgtggggagcaaacaggattagataccctggtagtccacgccgtaaacgatgaatgttagccgtcgggcagtatactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagctcttgacattcggggtatgggcattggagacgatgtccttcagttaggctggccccagaacaggtgctgcatggctgtcgtcagctcgtgtcgtgagatgttgggttaagtcccgcaacgagcgcaaccctcgcccttagttgccagcatttagttgggcactctaaggggactgccggtgataagccgagaggaaggtggggatgacgtcaagtcctcatggcccttacgggctgggctacacacgtgctacaatggtggtgacagtgggcagcgagacagcgatgtcgagctaatctccaaaagccatctcagttcggattgcactctgcaactcgagtgcatgaagttggaatcgctagtaatcgcagatcagcatgctgcggtgaatacgttcccgggccttgtacacaccgcccgtcacaccatgggagttggttttacccgaaggtagtgcgctaaccgcaaggaggcagctaaccacgtagttacgt

(19) United States
(12) Patent Application Publication
(10) Pub. No.: US 2023/0055742 A1
(43) Pub. Date: Feb. 23, 2023

(54) Strain of Beijerinckia Fluminensis and Application Thereof in Arsenic Oxidation

(71) Applicant: GUANGDONG INSTITUTE OF ECO-ENVIRONMENTAL SCIENCE & TECHNOLOGY, Guangzhou (CN)

(72) Inventors: Weimin Sun, Guangzhou (CN); Xiaoxu Sun, Guangzhou (CN); Baoqin Li, Guangzhou (CN); Yuqing Huang, Guangzhou (CN); Tianle Kong, Guangzhou (CN); Xiaoyu Wang, Guangzhou (CN); Miaomiao Zhang, Guangzhou (CN); Lang Qiu, Guangzhou (CN)

(21) Appl. No.: 17/790,406

(22) PCT Filed: Aug. 10, 2020
(86) PCT No.: PCT/CN2020/108071

§ 371 Date: Jun. 30, 2022

(30) Foreign Application Priority Data
Dec. 30, 2019 (CN) 201911389984.6

(51) Int. Cl. C12N 1/20; B09C 1/10

(52) U.S. Cl. CPC C12N 1/205; B09C 1/10; C12R 2001/01

(57) ABSTRACT

Provided are a strain ofBeijerinckia fluminensis and an application thereof in arsenic oxidation. The strain is AS-56, and has been deposited in the China Center for Type Culture Collection on 5 Dec. 2019, the deposit number being CCTCC NO: M 20191014. The strain AS-56 of Beijerinckia fluminensis can oxidise As(III) to the less toxic As(V) and can completely oxidize 0.15 g/L of As(III) solution to As(V), greatly reducing arsenic toxicity, and thus having wide prospects for application in environmental remediation.

### CROSS-REFERENCE TO RELATED APPLICATIONS

This application is the United States national phase of International Application No. PCT/CN2020/108071 filed Aug. 10, 2020, and claims priority to Chinese Patent Application No. 201911389984.6 filed Dec. 30, 2019, the disclosures of which are hereby incorporated by reference in their entireties.

### REFERENCE TO A SEQUENCE LISTING SUBMITTED VIA EFS-WEB

The content of the ASCII text file of the sequence listing named 08692_2204378_ST25.txt which is 2,854 bytes in size was created on Jun. 29, 2022 and electronically submitted via EFS-Web herewith the application is incorporated herein by reference in its entirety.

### BACKGROUND OF THE INVENTION

### Field of the Invention

The present invention belongs to a field of environmental microorganism, and in particular relates to a strain of Beijerinckia fluminensis, which can oxidize arsenic, thereby reducing the adverse effects of arsenic on the environment.

### Description of Related Art

In recent years, with the development of mining, chemical industry, pesticide and other industries, arsenic contamination of soil is very serious in some areas of Hunan, Guangdong, Guizhou, Yunnan and other provinces in China.

Arsenic is a typical carcinogen to human with high toxicity. At present, the treatment methods for arsenic-contaminated soil at home and abroad include physical, chemical and bioremediation methods. Physical methods such as electric remediation technology, soil replacement method and so on require a large amount of engineering work, a long time, and high treatment cost; chemical methods such as curing/stabilizing method, soil leaching method and so on have a narrow scope of use with strong limitations, and are prone to cause other environmental risks; and plant-remediation methods have a long treatment period, and differences in plants growing under different regional conditions make this technique difficult to be standardized. Meanwhile, the above methods are difficult to promote in the treatment of large areas of arsenic-contaminated soil.

Microbial remediation method is to subject contaminators to redox reaction, absorption, degradation and the like effects through indigenous microorganisms or artificially added functional microorganisms in the natural environment, by which the toxicity of contaminators in soil is reduced. It has the advantages of safety, high efficiency, low cost, environmental friendliness, capability of being applied in a large area and so on, which is a new technology for environmental remediation with the best development and application prospect.

Arsenic in the natural environment mainly exists in two valences: As(V) and As(III). Under most environmental conditions, the toxicity of As(III) is 25-60 times that of As(V). Therefore, if As(III) with higher toxicity can be oxidized to As(V) with lower toxicity, the mobility and toxicity of arsenic can be reduced, which has practical significance for environmental remediation.

Microorganisms in nature are widely involved in the geochemical cycle of arsenic, and they are highly adaptable to arsenic, some of which can even obtain energy for growth by oxidizing As(III) with As(III) in the environment oxidizided into As(V), reducing the toxicity of arsenic. Meanwhile, arsenic oxidation driven by microorganisms can significantly increase the oxidation rate of arsenic. Therefore, arsenic oxidation driven by microorganisms can be exploited to mitigate arsenic contamination in natural habitats.

The arsenic oxidation process driven by microorganisms can reduce the toxicity and mobility ability of arsenic in the environment, and play an important role in the remediation of arsenic contamination. The bacteria for arsenic oxidization found in the current research mainly belong to the genus Pseudomonas, Thiomonas, Bacillus, Achromobacter, etc. So far, any report about arsenic oxidation with Beijerinckia has not been found.

### SUMMARY OF THE INVENTION

An object of the present invention is to provide a strain AS-56 of Beijerinckia fluminensis, which has the function of oxidizing arsenic, may oxidize As(III) with higher toxicity to As(V) with lower toxicity, greatly reducing the toxicity of arsenic in the environment, and may be used in remediating an arsenic-contaminated environment such as mining area, etc.

The object of the present invention is realized by the following technical solutions:

A strain AS-56 of Beijerinckia fluminensis, which has been deposited in China Center for Type Culture Collection (address: at Wuhan University, Wuhan City, Hubei Province, China) on Dec. 5, 2019, and its deposit number is CCTCC NO: M 20191014.

A screening and isolation process of the strain AS-56 is: collecting arsenic-contaminated soil from tailings in realgar mining area in Shimen County, Hunan Province, culturing it in a liquid medium for one week, taking the cultural liquid and coating it evenly on a solid medium, placing it in a biochemical incubator to culture at 30° C., picking colonies in different forms and separating them through a streaking method after the bacteria grow out, and continuously separating and purifying to obtain a single colony, which is an efficient bacterium for arsenic oxidization with ability of oxidizing As(III).

An strain in the single colony is rod-shaped, Gram-negative, and 0.1-0.3×0.06-1.0 gm in size, and the bacterium is not motile. After identification by Biolog, it is found that the strain could metabolize 43 kinds of carbon sources such as acetic acid, D-gluconic acid, D-maltose, and so on, and shows tolerance to 8 kinds of substances such as 1% NaCl, troleandomycin, tetrazolium violet, and so on, in metabolism and tolerance tests of 94 different kinds of substances.

Combined with 16S rRNA and other related bioinformatics identification results, it is determined that the strain is Beijerinckia fluminensis, and it was named as Beijerinckia fluminensis AS-56.

By using high performance liquid chromatography-hydride generation-atomic fluorescence (HPLC-HG-AFS) technique to carry out quantitative analysis, it is found that the strain AS-56 in the present invention has ability of oxidizing arsenic and can oxidize As(III) to As(V).

Particularly, the strain AS-56 in the present invention can completely oxidize As(III) to As(V) in a solution containing 0.15 g/L As(III).

Particularly, the strain AS-56 in the present invention can be used to remediate arsenic-contaminated soil, and can oxidize As(III) therein to As(V).

Compared with the prior art, the present invention has the following advantages and effects:

The present invention discovers a strain AS-56 of Beijerinckia fluminensis which has ability of oxidizing arsenic; and this strain can oxidize As(III) to As(V) with lower toxicity, and can completely oxidize As(III) solution with a concentration of 0.15 g/L to As(V), greatly reducing the toxicity of arsenic. Therefore, this strain has broad application prospects in environmental remediation.

### BRIEF DESCRIPTION OF DRAWINGS

FIG. 1 is a scanning electron microscope photo of a strain AS-56 of Beijerinckia fluminensis.

FIG. 2 is a phylogenetic analysis of a strain AS-56 of Beijerinckia fluminensis.

FIG. 3 is a graph for oxidizing As(III) of a strain AS-56 of Beijerinckia fluminensis.

### DESCRIPTION OF THE INVENTION

The present invention will be further described in detail below in conjunction with examples and drawings, but embodiments of the present invention are limited thereto.

### EXAMLE 1

### Separation and Purification of a Strain

(1) Soil samples were collected from realgar tailings in Shimen County, Hunan Province. 1.5 g of arsenic-contaminated soil samples were taken into a 50 mL conical flask, and 15 mL of a liquid medium was added, the specific constituents of which were 20 g/L of sucrose, 1.0 g/L of K₂HPO₄, 0.5 g/L of MgSO₄, 0.5 g/L of NaCl, 0.1 g/L of FeSO₄, 0.005 g/L of MoNa₂O₄ and 2.0 g/L of CaCO₃. After inoculation, it was sealed with a sterilized aerobic membrane and placed in a shaker at 30 ° C. and 150 rpm for shaking culture.

(2) After culturing for one week, 20 μL of the cultural liquid was taken and coated evenly on a solid medium (15 g of agar was added per liter of the medium, and the other components were same as those of the liquid medium mentioned above), which was then placed at a biochemical incubator to be cultured at 30 ° C. After the bacteria grew out, colonies in different forms were picked and they were separated and purified through a streaking method, being inoculated in the solid medium by streaking, and this process was repeated for four times to obtain a single colony, which was then put in a refrigerator at 4° C. for later use, and preserved in a porcelain bead strain preservation tube at −80° C.

### EXAMLE 2

### Identification of the Strain

(1) Identification of Physiological and Biochemical Properties

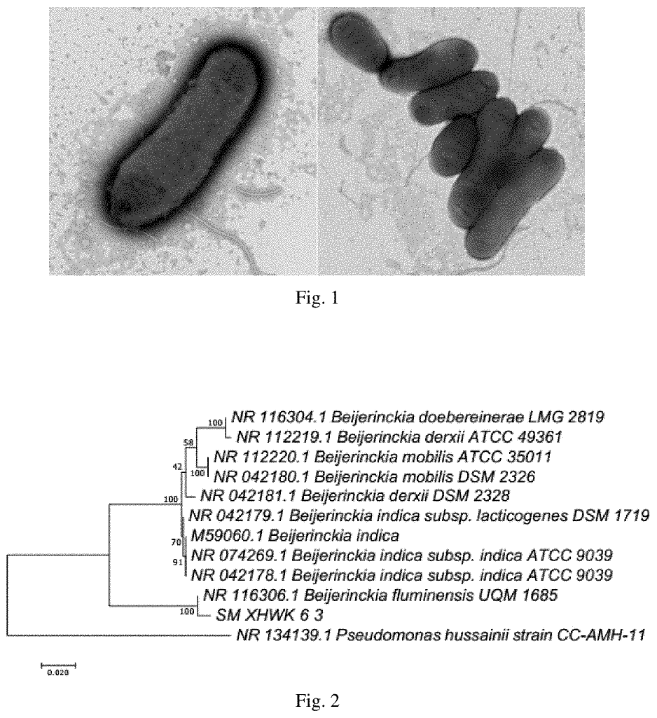

Scanning electron microscope observation was carried out on the single colony obtained in Example 1. Bacterial cells of this strain were rod-shaped and belonged to Brevibacterium, as shown in FIG. 1.

The strain was Gram-stained, and the specific steps were: droping a drop of water on a clean glass slide, picking bacterial cells with an inoculation ring and coating them evenly in the water, placing the glass slide close to the flame of an alcohol lamp to evaporate the water to dryness, adding ammonium oxalate crystal violet to the stain for 1 minute, rinsing with distilled water, adding iodine solution to cover the coated surface to stain for about 1 minute, rinsing with water and then absorbing water with an absorbent paper, adding a few drops of 95% alcohol, gently shaking the slide to decolorize, rinsing with water 20 seconds later, absorbing water, adding a safranine staining solution to stain for 1 minute, rinsing with distilled water, drying, and examining with a microscope. The staining result appears to be red, and it was confirmed that the strain was Gram-negative.

The steps of Biolog microbial identification were: after confirming that the strain is Gram-negative, picking a single purified colony and inoculating it into the Biolog special inoculum GN/GP-IF, to make a bacterial suspension with a certain cell concentration, and then transferring the bacterial suspension into 96 wells of the Biolog GEN III microwell identification plate in terms of 150 μL per well by using a pipette, marking it and then placing it in a constant-temperature biochemical incubator at 30 ° C. and culturing, reading characters of the metabolic fingerprints of the strains on a reader at 4-6 h and 16-24 h during culturing, and comparing the similarity in the Biolog database, to find the closest result.

Biolog database comparison steps were: enabling Biolog software, setting parameters such as culture time, strain name, strain number, strain type, and so on, wiping the bottom of the cultured identification plate with a clean tissue, putting it in the reader where A-1 hole is located at the upper left, clicking “Read This” to read, to get an identification result, as shown in Table 1.

As can be seen from Table 1, in the metabolism and tolerance tests of 94 different substances, the 1-9th columns are for metabolic tests, and the 10-12th columns are for tolerance tests. The results show that this strain can metabolize 43 kinds of carbon sources such as acetic acid, D-gluconic acid, D-maltose and so on, and show tolerance to 8 kinds of substances such as 1% NaCl, troleandomycin, tetrazolium violet and so on.

(2) Molecular Biological Identification

DNA was extracted from the single colony obtained in Example 1 by using a bacterial genomic DNA extraction kit, and then the universal primer pair of 16S rRNA gene was used to amplify and sequence F27 (5 ‘agagtttgatcmtggctcag3’) (SEQ. ID. NO.1) and 1492R (5 ‘ggytaccttgttacgactt3 ’) (SEQ. ID. NO.2). The obtained DNA sequence (SEQ. ID. NO.3) was imported into GenBank, and all sequences in the database were aligned and analyzed by the Blastn program.

A phylogenetic tree (as shown in FIG. 2) was constructed by using the 16S rRNA gene sequence, and it was found that the 16S rRNA of the single colony in Example 1 was clustered with Beijerinckia fluminensis, and the similarity reached 99%.

Based on the results of the above-described two aspects, it is determined that the strain isolated in Example 1 is Beiljerinckia fluminensis, which is named as Beijerinckia fluminensis AS-56.

### EXAMPLE 3

### Oxidative properties of the strain AS-56 to As(III)

An inoculated group and a non-inoculated group were set for the research on the oxidative properties of the strain AS-56 to As(III), and the specific operations were as follows:

The inoculated group: The AS-56 colonies on half of the surface of the plate were scraped from a solid medium and inoculated into a 50 mL penicillin bottle, and 25 mL of a liquid medium was added. The constituents of the medium were: 1.5 g/L of KH₂PO₄, 10.55 g/L of Na₂HPO₄.12H₂O, 0.3 g/L of NH₄Cl, 0.1 g/L of MgCl₂, 0.672 g/L of NaHCO₃, and 0.13 g/L of NaAsO₂. After inoculation, it was sealed with a rubber stopper and placed in a shaker at 30 ° C. for shaking culture at 150 rpm. Samples were taken respectively at 0 h, 6 h, 12 h, 24 h, and 48 h, and filtered and preserved at 4 ° C. for later use; after the samples were diluted to 500 times, concentrations of As(III) and As(V) therein were measured by high performance liquid chromatography-hydride generation-atomic fluorescence (HPLC-HG-AFS).

The non-inoculated group: 25 mL of the liquid medium was added into a 50 mL penicillin bottle, and the constituents of the medium were: 1.5 g/L of KH₂PO₄, 10.55 g/L of Na₂HPO₄.12H₂O, 0.3 g/L of NH₄Cl, 0.1 g/L of MgCl₂, 0.672 g/L of NaHCO₃, and 0.13 g/L of NaAsO₂. After inoculation, it was sealed with a rubber stopper and placed in a shaker at 30° C. for shaking culture at 150 rpm. Samples were taken respectively at 0 h, 6 h, 12 h, 24 h, and 48 h, and filtered and preserved at 4 ° C. for later use; after the samples were diluted to 500 times, concentrations of As(III) and As(V) therein were measured by high performance liquid chromatography-hydride generation-atomic fluorescence (HPLC-HG-AFS).

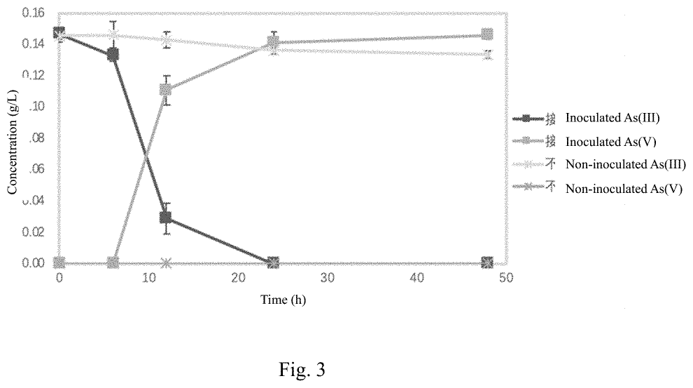

The concentrations of As(III) and As(V) from the inoculated group and the non-inoculated group were plotted as arsenic oxidation graph, as shown in FIG. 3. Wherein, X-axis is time (h), with time for sampling of 0 h, 6 h, 12 h, 24 h, and 48 h; and Y-axis is the concentration of arsenic (g/L).

It can be seen from FIG. 3 that the concentration of As(III) was basically maintained at about 0.14 g/L and the presence of As(V) could not be detected in the non-inoculated medium; however, the concentration of As(III) in the solution decreased continuously from 0.15 g/L to 0 g/L in the inoculated medium, while the concentration of As(V) rised from 0 g/L to about 0.15 g/L, indicating that the arsenic oxidation reaction occurred due to the inoculation of the strain, and inoculating the strain AS-56 of Beijerinckia fluminensis into the medium containing arsenic can oxidizing all As(III) to As(V).

The above-mentioned Examples are preferred embodiments of the present invention, but embodiments of the present invention are not limited by the above-mentioned Examples, and any other changes, modifications, substitutions, combinations, and simplifications made without departing from the spirit and principle of the present invention should be equivalent replacement modes, and are included within the protection scope of the present invention.

TABLE 1
Identification Results from Biolog GEN III Microbial Automatic Identification System

| | A1 | | | | | |
|---|---|---|---|---|---|---|
| Names of | Negative | A2 | A3 | A4 | A5 | A6 |
| strains | Control | Dextrin | D-Maltose | D-Trehalose | D-Cellobiose | Gentiobiose |
| 6-3 | − | − | + | + | + | + |
| | | B2 | | B4 | | B6 |
| | B1 | α-D- | B3 | β-Methy | B5 | N-Acetyl- |
| | D-Raffinose | Lactose | D-Melibiose | 1-D-Glucoside | D-Salicin | D-Glucosamine |
| 6-3 | + | + | + | + | + | + |
| | C1 | | | | C5 | |
| | α-D- | C2 | C3 | C4 | 3-Methyl | C6 |
| | Glucose | D-Mannose | D-Fructose | D-Galactose | Glucose | D-Fucose |
| 6-3 | + | + | + | + | − | + |
| | | | | | | D6 |
| | D1 | D2 | D3 | D4 | D5 | D-Glucose- |
| | D-Sorbitol | D-Mannitol | D-Arabitol | myo-Inositol | Glycerol | 6-PO4 |
| 6-3 | + | + | + | + | + | + |
| | | E2 | | | E5 | E6 |
| | E1 | Glycyl- | E3 | E4 | L-Aspartic | L-Glutamic |
| | Gelatin | L-Proline | L-Alanine | L-Arginine | Acid | Acid |
| 6-3 | − | w | + | + | + | + |
| | | F2 | F3 | F4 | F5 | |
| | F1 | D-Galacturonic | L-Galactonic | D-Gluconic | D-Glucuronic | F6 |
| | Pectin | Acid | Acid Lactone | Acid | Acid | Glucuronamide |
| 6-3 | + | − | − | + | + | − |
| | G1 | | | | | |
| | p-Hydroxy- | G2 | G3 | G4 | G5 | G6 |
| | Phenylacetic | Methyl | D-Lactic Acid | L-Lactic | Citric | α-Keto- |
| | Acid | Pyruvate | Methyl Ester | Acid | Acid | Glutaric Acid |
| 6-3 | − | − | − | − | − | − |
| | | | | H4 | | |
| | H1 | H2 | H3 | β-Hydroxy- | H5 | H6 |
| | Tween | γ-Amino- | α-Hydroxy- | D,LButyric | α-Keto- | Acetoacetic |
| | 40 | Butrytic Acid | Butyric Acid | Acid | Butyric Acid | Acid |
| 6-3 | − | − | − | − | − | + |

| | | | | A10 | | |
|---|---|---|---|---|---|---|
| Names of | A7 | A8 | A9 | Positive | A11 | A12 |
| strains | Sucrose | D-Turanose | Stachyose | Control | pH 6 | pH 5 |
| 6-3 | + | + | + | + | + | − |
| | B7 | B8 | B9 | | | |
| | N-Acetyl- | N-Acetyl- | N-Acetyl | B10 | B11 | B12 |
| | β- | D-Galac- | Neuraminic | 1% | 4% | 8% |
| | DMannosamine | tosamine | Acid | NaCl | NaCl | NaCl |
| 6-3 | w | + | − | + | − | − |
| | | | | C10 | | |
| | | | | 1% | C11 | |
| | C7 | C8 | C9 | Sodium | Fusidic | C12 |
| | L-Fucose | L-Rhamnose | Inosine | Lactate | Acid | D-Serine |
| 6-3 | + | + | − | w | − | − |
| | D7 | D8 | | D10 | D11 | D12 |
| | D-Fructose- | D-Aspartic | D9 | Trolean- | Rifamycin | Minocy- |
| | 6-PO4 | Acid | D-Serine | domycin | SV | cline |
| 6-3 | + | − | − | + | + | − |
| | | E8 | | | E11 | |
| | E7 | L-Pyroglutamic | E9 | E10 | Guanidine | E12 |
| | L-Histidine | Acid | L-Serine | Lincomycin | HCl | Niaproof 4 |
| 6-3 | + | + | + | + | w | − |
| | | | | | F11 | F12 |
| | F7 | F8 | F9 | | Tetrazo- | Tetrazo- |
| | Mucic | Quinic | D-Saccharic | F10 | lium | lium |
| | Acid | Acid | Acid | Vancomycin | Violet | Blue |
| 6-3 | − | + | − | + | + | + |
| | | | G9 | | | |
| | G7 | G8 | Bromo- | G10 | G11 | G12 |
| | D-Malic | L-Malic | Succinic | Nalidixic | Lithium | Potassium |
| | Acid | Acid | Acid | Acid | Chloride | Tellurite |
| 6-3 | − | + | + | − | w | w |
| | H7 | H8 | H9 | | H11 | H12 |
| | Propionic | Acetic | Formic | H10 | Sodium | Sodium |
| | Acid | Acid | Acid | Aztreonam | Butyrate | Bromate |
| 6-3 | − | + | − | + | − | − |

1. A strain AS-56 of Beijerinckia fluminensis, wherein it has been deposited in China Center for Type Culture Collection on Dec. 5, 2019, and its deposit number is CCTCC NO: M 20191014.

2. A method of oxidizing As(III) to AS(V) comprising oxidizing As(III) with the strain AS-56 of Beijerinckia fluminensis according to claim 1.

3. The method of claim 2, wherein the strain AS-56 oxidizes As(III) to As(V) in a solution containing 0.15 g/L As(III).

4. The method of claim 2, wherein the strain AS-56 is used to remediate arsenic-contaminated soil.
